(19) United States
(12) Patent Application Publication
(10) Pub. No.: US 2026/0294612 A1
(43) Pub. Date: Oct. 1, 2026

(54) ENDOPROSTHESIS FOR HEPATICOGASTROSTOMY USE

(71) Applicant: BOSTON SCIENTIFIC SCIMED, INC., Maple Grove, MN (US)

(72) Inventor: MARTYN G. FOLAN, Loughrea (IE)

(73) Assignee: BOSTON SCIENTIFIC SCIMED, INC., Maple Grove, MN (US)

(21) Appl. No.: 19/629,532

(22) Filed: Mar. 26, 2026

Related U.S. Application Data
(60) Provisional application No. 63/778,706, filed on Mar. 27, 2025.

(51) Int. Cl. A61F 2/04

(52) U.S. Cl. CPC A61F 2/04; A61F 2002/045; A61F 2220/0008; A61F 2230/005; A61F 2250/0039

(57) ABSTRACT

An endoprosthesis includes a body portion and a flared portion formed from a single wire. The single wire forms a first circumferential end segment extending circumferentially around the central axis at the first end perpendicular to the central axis, a second circumferential end segment extending circumferentially around the central axis at the second end perpendicular to the central axis, and a plurality of circumferential segments extending helically around the central axis between the first and second circumferential end segments. An endoprosthesis includes a body portion, a first end portion, and a flanged portion extending between the body and first end portions, wherein the body, first end, and flanged portions are formed from a single wire. An endoprosthesis includes a body portion and a plurality of loops extending radially outward from a first end, wherein the body portion and the plurality of loops are formed from a single wire.

### CROSS REFERENCE TO RELATED APPLICATIONS

The present application claims the benefit of US Provisional Patent Application Serial No. 63/778,706, filed Mar. 27, 2025, the disclosure of which is incorporated herein by reference.

### TECHNICAL FIELD

The present disclosure pertains to medical devices, methods for manufacturing medical devices, and uses thereof. More particularly, the present disclosure pertains to an endoprosthesis or stent for implantation in a body lumen, and associated methods.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, the biliary tract, etc. as well as in a variety of other applications in the body.

In some instances, it may be desirable to design an endoprosthesis to include sufficient flexibility and conformability to the body lumen, while maintaining sufficient radial force to open the body lumen at the treatment site and/or prevent migration of the endoprosthesis within the body lumen. In some instances, it may be desirable to reduce or limit foreshortening. In some instances, it may be desirable to reduce or limit migration. In some instances, different endoprosthesis configurations may provide different deliverability, flexibility, conformability, radial force/strength, and/or anchoring/migration characteristics.

Various medical procedures involve the temporary or permanent joining of non-connected anatomical structures. Some examples include a hepaticogastrostomy (HG) involving joining the hepatic duct and the stomach to drain the bile duct, a gastrojejeneum (GJ) bypass procedure to create an anastomosis between the small intestine and stomach wall, and stomas to create an artificial opening into the large intestine or other region of the digestive tract. In these medical procedures, peristalsis and gross organ movement in one or both of the anatomical structures being connected may create difficulties in using stents to join the structures due to stent migration or other complications.

Of the known medical devices and methods, each has certain advantages and disadvantages. There is an ongoing need to provide alternative medical devices as well as alternative methods for manufacturing and using medical devices.

### SUMMARY

In one example, an endoprosthesis having a first end and a second end may comprise a body portion extending axially along a central longitudinal axis, and a flared portion extending between the body portion and the first end. The body portion and the flared portion may be formed from a single wire extending from the first end to the second end. The single wire may form a first circumferential end segment extending circumferentially around the central longitudinal axis at the first end, wherein the first circumferential end segment is oriented generally perpendicular to the central longitudinal axis, a second circumferential end segment extending circumferentially around the central longitudinal axis at the second end, wherein the second circumferential end segment is oriented generally perpendicular to the central longitudinal axis, and a plurality of circumferential segments extending helically around the central longitudinal axis between the first circumferential end segment and the second circumferential end segment.

In addition, or alternatively, to any example disclosed herein, the endoprosthesis may comprise a transition portion extending from the body portion to the flared portion.

In addition, or alternatively, to any example disclosed herein, the transition portion angles radially outward from the body portion to the flared portion.

In addition, or alternatively, to any example disclosed herein, at least one circumferential segment of the plurality of circumferential segments disposed within the transition portion comprises a double circumferential loop of the single wire.

In addition, or alternatively, to any example disclosed herein, the body portion has a first outer diameter, and the flared portion has a second outer diameter greater than the first outer diameter.

In addition, or alternatively, to any example disclosed herein, the plurality of circumferential segments is oriented at an oblique angle relative to the central longitudinal axis, as viewed from a side of the endoprosthesis toward the central longitudinal axis.

In addition, or alternatively, to any example disclosed herein, the oblique angle is between about 70 degrees and about 85 degrees.

In addition, or alternatively, to any example disclosed herein, a removal suture is interwoven with the first circumferential end segment.

In addition, or alternatively, to any example disclosed herein, the plurality of circumferential segments has a first density within the body portion and a second density greater than the first density within the flared portion.

In addition, or alternatively, to any example disclosed herein, the endoprosthesis may comprise a polymeric covering fixedly attached to the body portion and the flared portion.

In addition, or alternatively, to any example disclosed herein, at least one circumferential segment of the plurality of circumferential segments disposed within the body portion comprises an anti-migration element projecting radially outward from the at least one circumferential segment of the plurality of circumferential segments disposed within the body portion, wherein the anti-migration element is formed from the single wire.

In addition, or alternatively, to any example disclosed herein, the first circumferential end segment comprises a double circumferential loop of the single wire.

In addition, or alternatively, to any example disclosed herein, the body portion is configured to be disposed within a first body lumen and the flared portion is configured to project into an interior space formed within a second body lumen such that the first end is spaced apart from a wall of the second body lumen.

In addition, or alternatively, to any example disclosed herein, the first body lumen is a duct of a biliary system of a patient and the second body lumen is a stomach of the patient.

In addition, or alternatively, to any example disclosed herein, and in a second example, an endoprosthesis having a first end and a second end may comprise a body portion extending axially along a central longitudinal axis from the second end toward the first end, a first end portion extending axially along a central longitudinal axis from the first end toward the second end, and a flanged portion extending between the body portion and the first end portion. The body portion, the first end portion, and the flanged portion may be formed from a single wire extending from the first end to the second end. The single wire may form a first circumferential end segment extending circumferentially around the central longitudinal axis at the first end, wherein the first circumferential end segment is oriented generally perpendicular to the central longitudinal axis, a second circumferential end segment extending circumferentially around the central longitudinal axis at the second end, wherein the circumferential second end segment is oriented generally perpendicular to the central longitudinal axis, and a plurality of circumferential segments extending helically around the central longitudinal axis between the first circumferential end segment and the second circumferential end segment.

In addition, or alternatively, to any example disclosed herein, the flanged portion may comprise a medial region having a medial outer diameter, wherein the medial outer diameter is greater than an outer diameter of the body portion and the medial outer diameter is greater than an outer diameter of the first end portion, a first transition portion extending from the body portion to the medial region, and a second transition portion extending from the first end portion to the medial region.

In addition, or alternatively, to any example disclosed herein, the endoprosthesis may comprise a polymeric covering fixedly attached to the body portion, the first end portion, and the flanged portion.

In addition, or alternatively, to any example disclosed herein, the body portion is configured to be disposed within a first body lumen and the first end portion is configured to project into an interior space formed within a second body lumen such that the first end is spaced apart from a wall of the second body lumen.

In addition, or alternatively, to any example disclosed herein, the body portion is configured to span a gap between the first body lumen and the second body lumen.

In addition, or alternatively, to any example disclosed herein, at least one circumferential segment of the plurality of circumferential segments disposed within the body portion comprises an anti-migration element projecting radially outward from the at least one circumferential segment of the plurality of circumferential segments disposed within the body portion, wherein the anti-migration element is formed from the single wire.

In addition, or alternatively, to any example disclosed herein, and in a third example, an endoprosthesis having a first end and a second end may comprise a body portion extending axially along a central longitudinal axis from the first end to the second end, and a plurality of loops extending radially outward from the first end. The body portion and the plurality of loops may be formed from a single wire extending from the first end to the second end. The single wire may form a first circumferential end segment extending circumferentially around the central longitudinal axis at the first end, wherein the first circumferential end segment includes the plurality of loops and is oriented generally perpendicular to the central longitudinal axis, a second circumferential end segment extending circumferentially around the central longitudinal axis at the second end, wherein the second circumferential end segment is oriented generally perpendicular to the central longitudinal axis, and a plurality of circumferential segments extending helically around the central longitudinal axis between the first circumferential end segment and the second circumferential end segment.

The above summary of some embodiments, aspects, and/or examples is not intended to describe each disclosed embodiment or every implementation of the present disclosure. The figures and detailed description which follow more particularly exemplify these embodiments .

### BRIEF DESCRIPTION OF THE DRAWINGS

The disclosure may be more completely understood in consideration of the following detailed description in connection with the accompanying drawings, in which:

FIG. 1 schematically illustrates selected portions of a patient’s anatomy;

FIG. 2 is a flat pattern view illustrating selected aspects of an endoprosthesis in accordance with the disclosure;

FIGS. 3-7 are side views illustrating selected aspects of an endoprosthesis in accordance with the disclosure;

FIG. 8 is a partial cutaway view illustrating selected aspects of an endoprosthesis in accordance with the disclosure disposed within first and second body lumens of a patient;

FIG. 9 is a side view illustrating selected aspects of an endoprosthesis in accordance with the disclosure; and

FIG. 10 is a side view illustrating selected aspects of an endoprosthesis in accordance with the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

In some instances, it may be desirable to provide an endoprosthesis, or stent, that can deliver luminal patency within the pancreaticobiliary tree of a patient. The relatively narrow biliary tract ducts consist of a series of bifurcations linking the liver, gallbladder, and pancreas via the papilla to the duodenal space for the transportation of bile and related enzymic substances for many metabolic functions but most commonly the body’s ability to digest and absorb fats and vitamins D and K. Endoscopic retrograde cholangiopancreatography (ERCP) may be used to diagnose and treat conditions of the bile ducts, including, for example, gallstones, inflammatory strictures, leaks (e.g., from trauma, surgery, etc.), and cancer. Blockage of the biliary duct may occur in many of the disorders of the biliary system, including the disorders of the liver, such as, primary schlerosing cholangitis, stone formation, scarring in the duct, etc. Draining blocked fluids from the biliary system may be used to treat the disorders. However, ERCP may not always be an option or may be unsuccessful in difficult anatomies, post-surgical anatomies, and/or challenging disease states. Hepaticogastrostomy (HG) may be used in cases where ERCP is not an option or fails. Other procedures, such as, but not limited to, choledochoduodenostomy (CDS) may be used to directly connect the common bile duct to the duodenum.

Hepaticogastrostomy Stenting (HGS) is a procedure that targets a hepatic duct within the liver directly from the stomach and places an endoprosthesis or stent creating an artificial pathway (bridging the peritoneal cavity) to facilitate ongoing internal biliary drainage. The HGS procedure is completed using an endoscopic ultrasound scope or other endoscope advanced through the esophagus that allows the endoscopist to “see” through the wall of the stomach and into the liver prior to gaining access. The endoscopist may position the scope with an appropriate clear trajectory to the hepatic duct. Guidewire access to the hepatic duct from the gastric region (e.g., the stomach) is then made using a needle followed by enlargement of the tract using a balloon and/or cystotome. Alternatively, access may be gained using a specialized all in one device, such as the Axios™ ‘Hot’ delivery device from Boston Scientific. The guidewire is advanced from the stomach into the duct. An endoprosthesis or stent is advanced and deployed to create and maintain a bridge between the hepatic duct and an interior of the stomach thus facilitating ongoing internal biliary drainage.

An additional factor endoscopists balance when positioning the endoscope for this procedure is also the proximal positioning of the endoprosthesis or stent within the stomach to minimize food impaction/migration into the lumen of the endoprosthesis or stent. Migration of the endoprosthesis or stent out of the stomach may be one of the highest severity risks with HGS. Migration of the endoprosthesis or stent out of the stomach can result in leakage and tissue damage (e.g., biliary peritonitis). Additionally, the migrated stent is free to abrade the outer gastric wall and other organs or vessels in the vicinity.

The hepatic duct and the stomach are spaced apart. The distance between the organs may vary and requires a relatively long stent. As the stomach muscles contract during digestion, the distance between the wall of the stomach relative to the hepatic duct varies, from a relatively small distance when the stomach is relaxed to a greater distance when the stomach is contracted. In addition to the wall of the stomach flexing, the stomach undergoes peristalsis during digestion. This relative motility of the stomach is understood to be complex, in three dimensions rather than in an exclusively linear manner. The distance between the target organs to be joined, as well as the relative movement of at least one of the organs, may increase the chance of stent migration.

An additional concern with traditional braided stents used in this indication is foreshortening which may cause deployment accuracy issues. The present disclosure is directed towards endoprostheses or stents for use in HGS which inhibit migration and/or inhibit or limit foreshortening.

While the present disclosure is described with respect to an HGS procedure, the devices, systems, and/or methods described herein may be used in stents, endoluminal implants, or transluminal implants where the proximal end terminates in the upper GI tract. This may include devices placed during a CDS procedure or during a successful ERCP procedure, among others. Further, while the present disclosure is described with respect to the pancreaticobiliary ductal system, the devices, systems, and/or methods described herein may be used in stents, endoluminal implants, or transluminal implants positioned in other parts of the body, such as, but not limited to, bodily tissue, bodily organs, vascular lumens, non-vascular lumens and combinations thereof, such as, but not limited to, in the coronary or peripheral vasculature, trachea, bronchi, colon, small intestine, esophagus, biliary tract, urinary tract, prostate, brain, stomach, and the like.

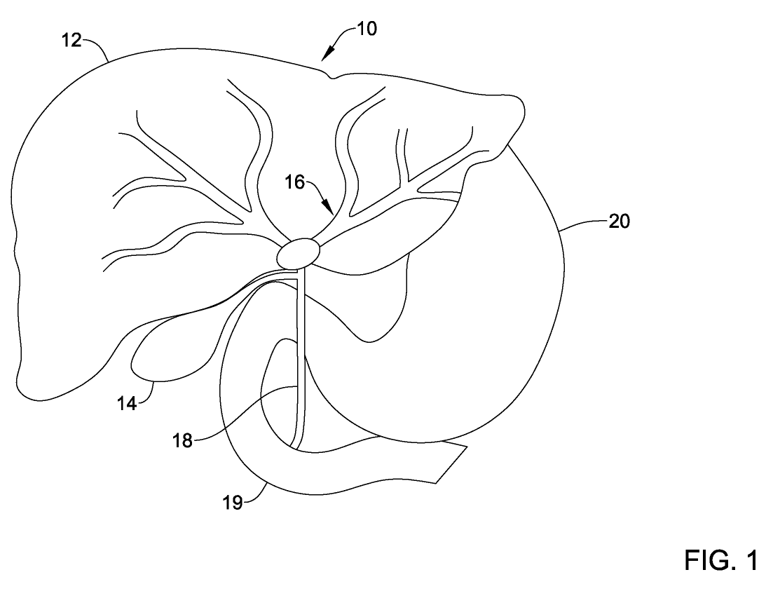

FIG. 1 schematically illustrates selected portions of a patient’s anatomy. The biliary system 10 may include, among other things given anatomical variations, the liver 12, the gall bladder 14, hepatic ducts 16, and the common bile duct 18. The common bile duct 18 connects to the duodenum 19 extending downstream from the stomach 20. In some cases, the common bile duct 18, or other lumens of the biliary system 10, may become blocked (partially or fully). In order to facilitate and/or restore biliary drainage, an HGS procedure may be performed to fluidly couple the hepatic ducts 16 to the stomach 20. As discussed herein, the stomach 20 and the liver 12 may move relative to each other within the peritoneal space during digestion and/or at other times as there is no direct attachment or connection between these two organs.

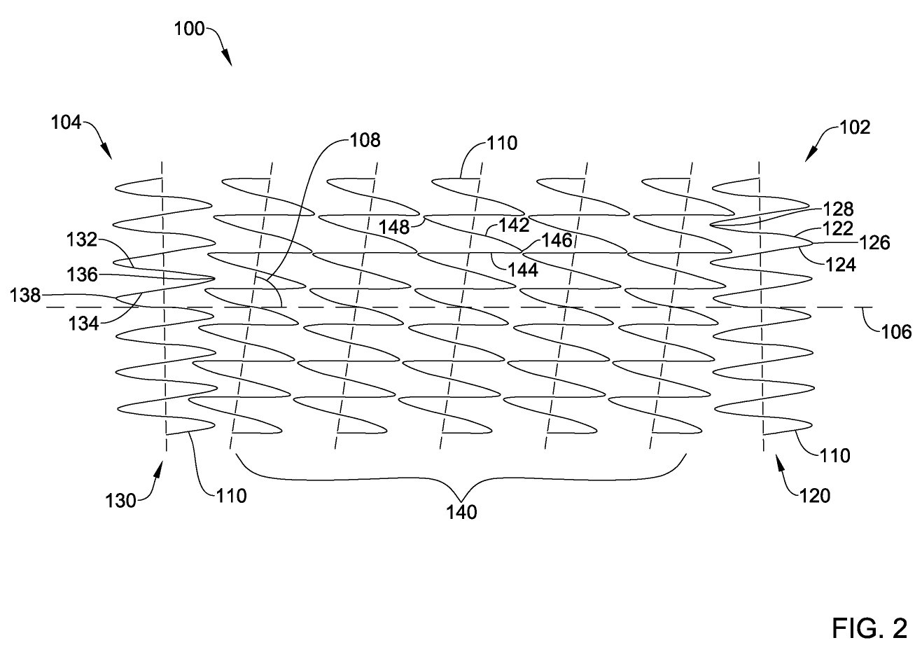

FIG. 2 schematically illustrates selected aspects related to the construction of an endoprosthesis 100, or a stent, in a flat pattern view. This may be referred to herein as a flat pattern configuration.

The endoprosthesis 100 may have a first end 102 and a second end 104. The endoprosthesis 100 may extend axially along a central longitudinal axis 106. In some embodiments, the endoprosthesis 100 may be formed from a single wire 110 extending from the first end 102 to the second end 104. In some embodiments, the first end 102 and the second end 104 may define a length of the endoprosthesis 100 and/or an overall length of the endoprosthesis 100. In at least some embodiments, the endoprosthesis 100 may be tubular. The endoprosthesis 100 may define a lumen extending therethrough from the first end 102 to the second end 104. The single wire 110 may extend circumferentially around the central longitudinal axis 106 and/or the single wire 110 may encircle the central longitudinal axis 106. The single wire 110 is and/or consists of only one wire.

Some suitable but non-limiting examples of materials for the endoprosthesis 100 and/or the single wire 110, such as metallic materials, composite materials, shape memory materials, combinations thereof, etc., are discussed below. In one non-limiting example, the endoprosthesis 100 and/or the single wire 110 may be formed from nickel-titanium alloy (e.g., nitinol). Other configurations and/or materials are also contemplated.

In some embodiments, the single wire 110 may form a first circumferential end segment 120 extending circumferentially around the central longitudinal axis 106 at and/or proximate the first end 102. The first circumferential end segment 120 may comprise an undulating arrangement (e.g., a sinusoidal arrangement) of first struts 122 and second struts 124 defining peaks 126 and valleys 128. In at least some embodiments, the first struts 122 of first circumferential end segment 120 have a first length defined by the peaks 126 and the valleys 128 and the second struts 124 of first circumferential end segment 120 have a second length defined by the peaks 126 and the valleys 128, wherein the first length of the first struts 122 is equal to the second length of the second struts 124. In some embodiments, the first length of the first struts 122 of first circumferential end segment 120 and the second length of the second struts 124 of first circumferential end segment 120 may be uniform and/or constant within the first circumferential end segment 120. In some alternative embodiments, the first length of the first struts 122 of first circumferential end segment 120 and the second length of the second struts 124 of first circumferential end segment 120 may vary within the first circumferential end segment 120.

In some embodiments, the first length of the first struts 122 of first circumferential end segment 120 and/or the second length of the second struts 124 of first circumferential end segment 120 may be between about 1 millimeter and about 15 millimeters. In some embodiments, the first length of the first struts 122 of first circumferential end segment 120 and/or the second length of the second struts 124 of first circumferential end segment 120 may be between about 3 millimeters and about 12 millimeters. In some embodiments, the first length of the first struts 122 of first circumferential end segment 120 and/or the second length of the second struts 124 of first circumferential end segment 120 may be between about 5 millimeters and about 10 millimeters. Other configurations are also contemplated.

In at least some embodiments, the first circumferential end segment 120 may be oriented generally perpendicular to the central longitudinal axis 106. In some embodiments, each strut (e.g., the first struts 122, the second struts 124) of the first circumferential end segment 120 may comprise and/or define a centroid disposed equidistantly between the peaks 126 and the valleys 128. The centroid of each strut (e.g., the first struts 122, the second struts 124) of the first circumferential end segment 120 may lie in a plane oriented perpendicular to the central longitudinal axis 106. In some embodiments, the peaks 126 of the first circumferential end segment 120 may touch and/or lie in a plane oriented perpendicular to the central longitudinal axis 106. In some embodiments, the valleys 128 of the first circumferential end segment 120 may touch and/or lie in a plane oriented perpendicular to the central longitudinal axis 106.

In some embodiments, the single wire 110 may form a second circumferential end segment 130 extending circumferentially around the central longitudinal axis 106 at and/or proximate the second end 104. The second circumferential end segment 130 may comprise an undulating arrangement (e.g., a sinusoidal arrangement) of first struts 132 and second struts 134 defining peaks 136 and valleys 138. In at least some embodiments, the first struts 132 of the second circumferential end segment 130 have a first length defined by the peaks 136 and the valleys 138 and the second struts 134 of the second circumferential end segment 130 have a second length defined by the peaks 136 and the valleys 138, wherein the first length of the first struts 132 of the second circumferential end segment 130 is equal to the second length of the second struts 134 of the second circumferential end segment 130. In some embodiments, the first length of the first struts 132 of the second circumferential end segment 130 and the second length of the second struts 134 of the second circumferential end segment 130 may be uniform and/or constant within the second circumferential end segment 130. In some alternative embodiments, the first length of the first struts 132 of the second circumferential end segment 130 and the second length of the second struts 134 of the second circumferential end segment 130 may vary within the second circumferential end segment 130.

In some embodiments, the first length of the first struts 132 of the second circumferential end segment 130 and/or the second length of the second struts 134 of the second circumferential end segment 130 may be between about 1 millimeter and about 15 millimeters. In some embodiments, the first length of the first struts 132 of the second circumferential end segment 130 and/or the second length of the second struts 134 of the second circumferential end segment 130 may be between about 3 millimeters and about 12 millimeters. In some embodiments, the first length of the first struts 132 of the second circumferential end segment 130 and/or the second length of the second struts 134 of the second circumferential end segment 130 may be between about 5 millimeters and about 10 millimeters. Other configurations are also contemplated.

In at least some embodiments, the second circumferential end segment 130 may be oriented generally perpendicular to the central longitudinal axis 106. In some embodiments, each strut (e.g., the first struts 132, the second struts 134) of the second circumferential end segment 130 may comprise and/or define a centroid disposed equidistantly between the peaks 136 and the valleys 138. The centroid of each strut (e.g., the first struts 132, the second struts 134) of the second circumferential end segment 130 may lie in a plane oriented perpendicular to the central longitudinal axis 106. In some embodiments, the peaks 136 of the second circumferential end segment 130 may touch and/or lie in a plane oriented perpendicular to the central longitudinal axis 106. In some embodiments, the valleys 138 of the second circumferential end segment 130 may touch and/or lie in a plane oriented perpendicular to the central longitudinal axis 106.

In some embodiments, the single wire 110 may form a plurality of circumferential segments 140 extending helically around the central longitudinal axis 106 between the first circumferential end segment 120 and the second circumferential end segment 130. In some embodiments, the plurality of circumferential segments 140 may comprise two circumferential segments, three circumferential segments, four circumferential segments, etc. up to a desired number of circumferential segments that combine with the first circumferential end segment 120 and the second circumferential end segment 130 to produce a desired overall length for the endoprosthesis 100.

In some embodiments, each circumferential segment of the plurality of circumferential segments 140 may comprise an undulating arrangement (e.g., a sinusoidal arrangement) of first struts 142 and second struts 144 defining peaks 146 and valleys 148. In at least some embodiments, the first struts 142 of each circumferential segment of the plurality of circumferential segments 140 have a first length defined by the peaks 146 and the valleys 148 and the second struts 144 of each circumferential segment of the plurality of circumferential segments 140 have a second length defined by the peaks 146 and the valleys 148, wherein the first length of the first struts 142 of each circumferential segment of the plurality of circumferential segments 140 is equal to the second length of the second struts 144 of each circumferential segment of the plurality of circumferential segments 140. In some embodiments, the first length of the first struts 142 of each circumferential segment of the plurality of circumferential segments 140 and the second length of the second struts 144 of each circumferential segment of the plurality of circumferential segments 140 may be uniform and/or constant within each circumferential segment of the plurality of circumferential segments 140. In some alternative embodiments, the first length of the first struts 142 of each circumferential segment of the plurality of circumferential segments 140 and the second length of the second struts 144 of each circumferential segment of the plurality of circumferential segments 140 may vary within each circumferential segment of the plurality of circumferential segments 140.

In some embodiments, the first length of the first struts 142 of each circumferential segment of the plurality of circumferential segments 140 and/or the second length of the second struts 144 of each circumferential segment of the plurality of circumferential segments 140 may be between about 1 millimeter and about 15 millimeters. In some embodiments, the first length of the first struts 142 of each circumferential segment of the plurality of circumferential segments 140 and/or the second length of the second struts 144 of each circumferential segment of the plurality of circumferential segments 140 may be between about 3 millimeters and about 12 millimeters. In some embodiments, the first length of the first struts 142 of each circumferential segment of the plurality of circumferential segments 140 and/or the second length of the second struts 144 of each circumferential segment of the plurality of circumferential segments 140 may be between about 5 millimeters and about 10 millimeters. Other configurations are also contemplated.

In some embodiments, the plurality of circumferential segments 140 may extend helically around the central longitudinal axis 106 from the first circumferential end segment 120 to the second circumferential end segment 130. In some embodiments, each strut (e.g., the first struts 142, the second struts 144) of each circumferential segment of the plurality of circumferential segments 140 may comprise and/or define a centroid disposed equidistantly between the peaks 146 and the valleys 148.

In some embodiments, the centroids of each strut (e.g., the first struts 142, the second struts 144) of each circumferential segment of the plurality of circumferential segments 140 may extend and/or may be arranged helically around the central longitudinal axis 106 from the first circumferential end segment 120 to the second circumferential end segment 130. In some embodiments, the peaks 146 may extend and/or may be arranged helically around the central longitudinal axis 106 from the first circumferential end segment 120 to the second circumferential end segment 130. In some embodiments, the valleys 148 may extend and/or may be arranged helically around the central longitudinal axis 106 from the first circumferential end segment 120 to the second circumferential end segment 130.

In some embodiments, the centroid of each strut (e.g., the first struts 142, the second struts 144) of each circumferential segment of the plurality of circumferential segments 140 may lie in a plane oriented at an oblique angle 108 relative to the central longitudinal axis 106, as viewed in the flat pattern configuration and/or as viewed from a side of the endoprosthesis 100 toward the central longitudinal axis 106. In some embodiments, the peaks 146 of each circumferential segment of the plurality of circumferential segments 140 may touch and/or lie in a plane oriented at an oblique angle relative to the central longitudinal axis 106, as viewed in the flat pattern configuration and/or as viewed from a side of the endoprosthesis 100 toward the central longitudinal axis 106. In some embodiments, the valleys 148 of each circumferential segment of the plurality of circumferential segments 140 may touch and/or lie in a plane oriented at an oblique angle relative to the central longitudinal axis 106, as viewed in the flat pattern configuration and/or as viewed from a side of the endoprosthesis 100 toward the central longitudinal axis 106.

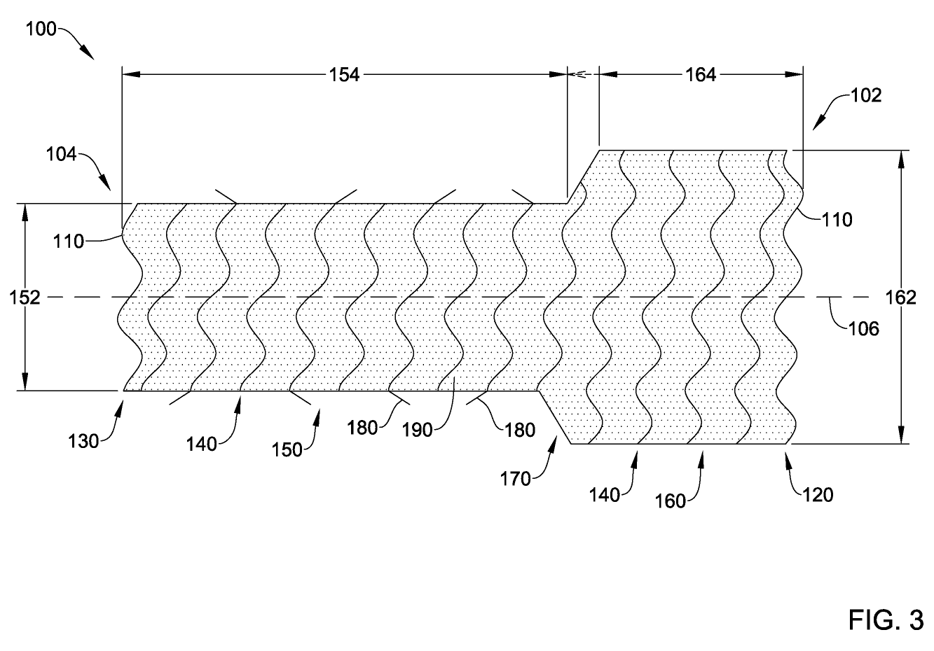

In some embodiments, the plurality of circumferential segments 140 may be oriented at the oblique angle 108 relative to the central longitudinal axis 106, as viewed in the flat pattern configuration and/or as viewed from a side of the endoprosthesis 100 toward the central longitudinal axis 106 (e.g., FIG. 3). In some embodiments, the oblique angle 108 may be between about 45 degrees and about 85 degrees. In some embodiments, the oblique angle 108 may be between about 60 degrees and about 85 degrees. In some embodiments, the oblique angle 108 may be between about 70 degrees and about 85 degrees. In one non-limiting example, the oblique angle 108 may be about 79 degrees. Other configurations are also contemplated.

In some embodiments, the peaks 146 and the valleys 148 of adjacent circumferential segments of the plurality of circumferential segments 140 may extend and/or may be aligned helically along the length of the endoprosthesis 100. In some embodiments, the peaks 146 and the valleys 148 of adjacent circumferential segments of the plurality of circumferential segments 140 may extend and/or may be aligned helically along and/or around the central longitudinal axis 106. In some embodiments, the peaks 146 and the valleys 148 of each circumferential segment of the plurality of circumferential segments 140 may extend and/or may be aligned helically along the length of the endoprosthesis 100. In some embodiments, the peaks 146 and the valleys 148 of each circumferential segment of the plurality of circumferential segments 140 may extend and/or may be aligned helically along and/or around the central longitudinal axis 106. Other configurations are also contemplated.

In some embodiments, the peaks 146 and the valleys 148 of adjacent circumferential segments of the plurality of circumferential segments 140 may be aligned with each other at an oblique angle relative to the central longitudinal axis 106 in the flat pattern configuration along the length of the endoprosthesis 100. In some embodiments, the peaks 146 and the valleys 148 of each circumferential segment of the plurality of circumferential segments 140 may be aligned with each other at an oblique angle relative to the central longitudinal axis 106 in the flat pattern configuration along the length of the endoprosthesis 100. For example, the peaks 146 of one circumferential segment of the plurality of circumferential segments 140 may be aligned at an oblique angle relative to the central longitudinal axis 106 in the flat pattern configuration with the peaks 146 of an immediately adjacent circumferential segment of the plurality of circumferential segments 140. Similarly, the valleys 148 of one circumferential segment of the plurality of circumferential segments 140 may be aligned at an oblique angle relative to the central longitudinal axis 106 in the flat pattern configuration with the valleys 148 of an immediately adjacent circumferential segment of the plurality of circumferential segments 140. Other configurations are also contemplated.

In some embodiments, the plurality of circumferential segments 140 may be oriented substantially parallel to each other in the flat pattern configuration. For example, in the flat pattern configuration, planes containing centroids of each circumferential segment of the plurality of circumferential segments 140 may be oriented parallel to each other. In another example, in the flat pattern configuration, planes containing and/or touching the peaks 146 of each circumferential segment of the plurality of circumferential segments 140 may be oriented parallel to each other. In another example, in the flat pattern configuration, planes containing and/or touching the valleys 148 of each circumferential segment of the plurality of circumferential segments 140 may be oriented parallel to each other.

In some embodiments, each adjacent pair of circumferential segments of the plurality of circumferential segments 140 defines an axial spacing therebetween. The axial spacing may be measured perpendicular to a plane or planes containing the centroids of one or both circumferential segments of each adjacent pair of circumferential segments of the plurality of circumferential segments 140 in the flat pattern configuration. In some embodiments, the axial spacing may be defined between the valleys 148 of one circumferential segment and the peaks 146 of an immediately adjacent circumferential segment in the flat pattern configuration. For the purpose of this disclosure, the axial spacing shall be understood to not include any axial overlap with the first struts 142 and/or the second struts 144 of any given circumferential segment in the flat pattern configuration.

In some embodiments, the axial spacing between adjacent pairs of circumferential segments of the plurality of circumferential segments 140 may be uniform along the length of the endoprosthesis 100 in the flat pattern configuration. In some embodiments, adjacent pairs of circumferential segments of the plurality of circumferential segments 140 are equally and/or uniformly spaced apart along the length of the endoprosthesis 100 in the flat pattern configuration. In some embodiments, the axial spacing between adjacent pairs of circumferential segments of the plurality of circumferential segments 140 may vary along the length of the endoprosthesis 100. In some embodiments, adjacent pairs of circumferential segments of the plurality of circumferential segments 140 are unequally and/or non-uniformly spaced apart along the length of the endoprosthesis 100. In some embodiments, the axial spacing between adjacent pairs of circumferential segments of the plurality of circumferential segments 140 may increase along the length of the endoprosthesis 100 from the first end 102 toward and/or to the second end 104. In some embodiments, the axial spacing between adjacent pairs of circumferential segments of the plurality of circumferential segments 140 may decrease along the length of the endoprosthesis 100 from the first end 102 toward and/or to the second end 104. Other configurations are also contemplated.

In some embodiments, the peaks 146 and the valleys 148 of each circumferential segment of the plurality of circumferential segments 140 may define a circumferential segment height for that circumferential segment measured perpendicular to a first plane containing and/or touching the peaks 146 and a second plane containing and/or touching the valleys 148 within a given circumferential segment of the plurality of circumferential segments 140 in the flat pattern configuration. In some embodiments, the circumferential segment height may be uniform along the length of the endoprosthesis 100 and/or among the plurality of circumferential segments 140. In some embodiments, the circumferential segment height may vary along the length of the endoprosthesis 100 and/or among the plurality of circumferential segments 140. In some embodiments, the circumferential segment height may increase along the length of the endoprosthesis 100 and/or among the plurality of circumferential segments 140 from the first end 102 toward and/or to the second end 104. In some embodiments, the circumferential segment height may decrease along the length of the endoprosthesis 100 and/or among the plurality of circumferential segments 140 from the first end 102 toward and/or to the second end 104.

In some embodiments, the first circumferential end segment 120 may comprise a greater quantity of peaks 126 and valleys 128 than the second circumferential end segment 130 has peaks 136 and valleys 138 and/or the plurality of circumferential segments 140 has peaks 146 and valleys 148. Such a configuration may increase outward radial force within the first circumferential end segment 120 compared to the second circumferential end segment 130and/or the plurality of circumferential segments 140.

In some embodiments, the second circumferential end segment 130 may comprise a greater quantity of peaks 136 and valleys 138 than the first circumferential end segment 120 has peaks 126 and valleys 128 and/or the plurality of circumferential segments 140 has peaks 146 and valleys 148. Such a configuration may increase outward radial force within the second circumferential end segment 130 compared to the first circumferential end segment 120 and/or the plurality of circumferential segments 140.

In some embodiments, the plurality of circumferential segments 140 may comprise a greater quantity of peaks 146 and valleys 148 than the first circumferential end segment 120 has peaks 126 and valleys 128 and/or the second circumferential end segment 130 has peaks 136 and valleys 138. Such a configuration may increase outward radial force within the plurality of circumferential segments 140 compared to the first circumferential end segment 120 and/or the second circumferential end segment 130. In some embodiments, selected circumferential segments within the plurality of circumferential segments 140 may comprise a greater quantity of peaks 146 and valleys 148 than other circumferential segments within the plurality of circumferential segments 140, thereby varying outward radial force along and/or within the plurality of circumferential segments 140. For example, the endoprosthesis 100 may be configured such that selected and/or specific locations may have increased outward radial force. Other configurations are also contemplated.

FIGS. 3-7 schematically illustrate selected aspects of example configurations of the endoprosthesis 100 in a side view. It shall be understood that while various aspects may be shown in different example configurations disclosed herein, configurations are contemplated within the scope of the disclosure that combine various aspects from different example configurations unless explicitly stated to the contrary.

The endoprosthesis 100 may be configured to shift between a delivery configuration and a deployed configuration. In some embodiments, the endoprosthesis 100 may be configured to be disposed within a delivery system and/or a delivery sheath in the delivery configuration. In some embodiments, the endoprosthesis 100 may be configured to be disposed between an inner tubular member and an outer tubular member of the delivery system and/or the delivery sheath in the delivery configuration. In some embodiments, the inner tubular member and the outer tubular member may be configured to translate relative to each other to deploy the endoprosthesis 100. Other configurations are also contemplated.

The endoprosthesis 100 may be configured to shift toward and/or to the deployed configuration when unconstrained. In some embodiments, the endoprosthesis 100 may be configured to self-expand from the delivery configuration to the deployed configuration. In some embodiments, the endoprosthesis 100 may be self-biased toward the deployed configuration. In some embodiments, the endoprosthesis 100 may be balloon expandable from the delivery configuration to the deployed configuration. Other configurations are also contemplated. In at least some embodiments, the endoprosthesis 100 may be radially expanded in the deployed configuration compared to the delivery configuration.

In some embodiments, the endoprosthesis 100 may comprise a body portion 150 extending axially along the central longitudinal axis 106. In some embodiments, the endoprosthesis 100 may comprise a flared portion 160 extending between the body portion 150 and the first end 102, as seen in FIG. 3. In some embodiments, the body portion 150 and the flared portion 160 may be formed from the single wire 110 extending from the first end 102 to the second end 104. As discussed herein, the single wire 110 may form the first circumferential end segment 120, the second circumferential end segment 130, and the plurality of circumferential segments 140.

In some embodiments, the endoprosthesis 100 may comprise a transition portion 170 extending from the body portion 150 to the flared portion 160. The transition portion 170 may be formed from the single wire 110. The transition portion 170 may angle radially outward from the body portion 150 toward and/or to the flared portion 160, as seen in FIG. 3, in the deployed configuration of the endoprosthesis 100. In some embodiments, the transition portion 170 may comprise at least one circumferential segment of the plurality of circumferential segments 140. In some embodiments, the transition portion 170 may comprise two or more circumferential segments of the plurality of circumferential segments 140.

In some embodiments, the body portion 150 may have a first outer diameter 152. In some embodiments, the first outer diameter 152 of the body portion 150 may be generally uniform and/or generally constant along the length of the body portion 150. In some embodiments, the first outer diameter 152 of the body portion 150 may be between about 2 millimeters and about 30 millimeters, between about 4 millimeters and about 25 millimeters, between about 6 millimeters and about 20 millimeters, between about 8 millimeters and about 15 millimeters, etc. In one non-limiting example, the first outer diameter 152 of the body portion 150 may be about 10 millimeters. Other configurations are also contemplated.

In some embodiments, the flared portion 160 may have a second outer diameter 162. In some embodiments, the second outer diameter 162 of the flared portion 160 may be generally uniform and/or generally constant along the length of the flared portion 160. The second outer diameter 162 may be greater than the first outer diameter 152. In some embodiments, the second outer diameter 162 may be about 20% greater than the first outer diameter 152. In some embodiments, the second outer diameter 162 may be about 30% greater than the first outer diameter 152. In some embodiments, the second outer diameter 162 may be about 40% greater than the first outer diameter 152. In some embodiments, the second outer diameter 162 may be about 50% greater than the first outer diameter 152. Other configurations are also contemplated. In some embodiments, the second outer diameter 162 of the flared portion 160 may be between about 2.2 millimeters and about 45 millimeters, between about 4.8 millimeters and about 37.5 millimeters, between about 7.2 millimeters and about 30 millimeters, between about 9.6 millimeters and about 22.5 millimeters, etc. In one non-limiting example, the second outer diameter 162 of the flared portion 160 may be about 14 millimeters. Other configurations are also contemplated.

In some embodiments, the overall length of the endoprosthesis 100 may be about 100 millimeters to about 150 millimeters. In some embodiments, the overall length of the endoprosthesis 100 may be about 110 millimeters to about 140 millimeters. In some embodiments, the overall length of the endoprosthesis 100 may be about 120 millimeters to about 130 millimeters. Other configurations are also contemplated.

In some embodiments, the endoprosthesis 100 may be configured to minimize and/or avoid foreshortening and/or a change in the overall length of the endoprosthesis 100 as or the endoprosthesis 100 shifts between the delivery configuration and the deployed configuration. In some embodiments, the overall length of the endoprosthesis 100 from the first end 102 to the second end 104 may be configured to change by less than 10% when shifting between the delivery configuration and the deployed configuration. In some embodiments, the overall length of the endoprosthesis 100 from the first end 102 to the second end 104 may be configured to change by less than 7.5% when shifting between the delivery configuration and the deployed configuration. In some embodiments, the overall length of the endoprosthesis 100 from the first end 102 to the second end 104 may be configured to change by less than 5% when shifting between the delivery configuration and the deployed configuration. In some embodiments, the overall length of the endoprosthesis 100 from the first end 102 to the second end 104 may be configured to change by less than 2.5% when shifting between the delivery configuration and the deployed configuration. In some embodiments, the overall length of the endoprosthesis 100 from the first end 102 to the second end 104 may be configured to change by less than 1% when shifting between the delivery configuration and the deployed configuration. Other configurations are also contemplated.

In some embodiments, the body portion 150 may have a body length 154 of about 80 millimeters to about 120 millimeters. In some embodiments, the body length 154 may be about 90 millimeters to about 110 millimeters. In one non-limiting example, the body length 154 may be about 100 millimeters. Other configurations are also contemplated.

In some embodiments, the flared portion 160 may have a flare length 164 of about 12 millimeters to about 30 millimeters. In some embodiments, the flare length 164 may be about 15 millimeters to about 25 millimeters. In some embodiments, the flare length 164 may be about 18 millimeters to about 22 millimeters. In some embodiments, the flare length 164 may include the transition portion 170. In one non-limiting example, the flare length 164, including the transition portion 170, may be about 20 millimeters. Other configurations are also contemplated.

In some embodiments, the endoprosthesis 100 and/or the body portion 150 may comprise one or more anti-migration elements disposed along the body length 154 of the endoprosthesis 100 and/or the body portion 150. In some embodiments, at least one circumferential segment of the plurality of circumferential segments 140 may comprise an anti-migration element 180 projecting radially outward from the at least one circumferential segment of the plurality of circumferential segments 140 and/or an outer surface of the endoprosthesis 100 and/or the body portion 150. In some embodiments, at least one circumferential segment of the plurality of circumferential segments 140 may comprise a plurality of anti-migration elements projecting radially outward from the at least one circumferential segment of the plurality of circumferential segments 140 and/or the outer surface of the endoprosthesis 100 and/or the body portion 150. The one or more anti-migration elements and/or the anti-migration element 180 of the at least one circumferential segment of the plurality of circumferential segments 140 may be formed from the single wire 110. For example, each anti-migration element may be formed from a peak 146 (e.g., FIG. 2) or a valley 148 (e.g., FIG. 2) that may be deflected, bent, and/or formed to extend radially outward from the at least one circumferential segment of the plurality of circumferential segments 140 and/or the outer surface of the endoprosthesis 100 and/or the body portion 150.

In some embodiments, at least one anti-migration element may project radially outward and toward the second end 104 (e.g., at a non-zero angle relative to the outer surface of the endoprosthesis 100 and/or the body portion 150) and/or at least one anti-migration element may project radially outward and toward the first end 102 (e.g., at a non-zero angle relative to the outer surface of the endoprosthesis 100 and/or the body portion 150). Other configurations are also contemplated.

The endoprosthesis 100 may comprise a polymeric covering 190, shown in the figures via dotted shading, coupled to the body portion 150 and the flared portion 160 and the transition portion 170, and/or the single wire 110. In some embodiments, the polymeric covering 190 may be fixedly attached to the body portion 150 and the flared portion 160 and the transition portion 170, and/or the single wire 110, and/or the plurality of circumferential segments 140.

In some embodiments, the polymeric covering 190 may extend along an inner surface of the endoprosthesis 100, the single wire 110, and/or the body portion 150, the flared portion 160, and the transition portion 170. In some embodiments, the polymeric covering 190 may extend along an outer surface of the endoprosthesis 100, the single wire 110, and/or the body portion 150, the flared portion 160, and the transition portion 170. In at least some embodiments, the single wire 110, and/or the body portion 150, the flared portion 160, and the transition portion 170 may be embedded within the polymeric covering 190. In some embodiments, the anti-migration element 180, the plurality of anti-migration elements, etc. may be covered by and/or embedded within the polymeric covering 190. Other configurations, including combinations thereof, are also contemplated. Some suitable but non-limiting examples of polymeric materials for the polymeric covering 190 are discussed below.

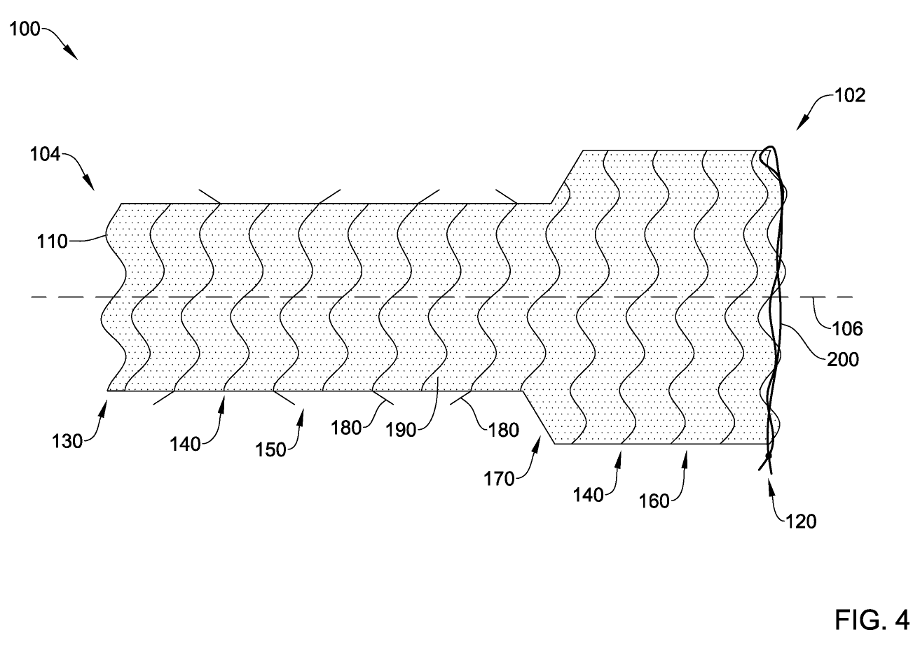

In some embodiments, the endoprosthesis 100 may comprise a removal suture 200 disposed proximate the first end 102. The removal suture 200 may be configured to facilitate and/or aid removal of the endoprosthesis 100 from the patient after deployment. In some embodiments, the removal suture 200 may be interwoven with the first end 102 and/or the first circumferential end segment 120, as seen in FIG. 4. In some embodiments, the removal suture 200 may be at least partially wrapped around the single wire 110 within the first circumferential end segment 120. In some embodiments, the removal suture 200 may be configured to be grasped and/or held, such as with a forceps or other grasping tool. In some embodiments, the removal suture 200 may be configured to transfer tension applied thereto to the endoprosthesis 100, the first end 102, and/or the first circumferential end segment 120 to facilitate removing the endoprosthesis 100 from the patient after deployment. In some embodiments, the removal suture 200 may be partially embedded within the polymeric covering 190. In some embodiments, the removal suture 200 may be passed through the polymeric covering 190 as it is interwoven with the first circumferential end segment 120. In some embodiments, the removal suture 200 may also be interwoven with one or more circumferential segments of the plurality of circumferential segments 140. Other configurations are also contemplated.

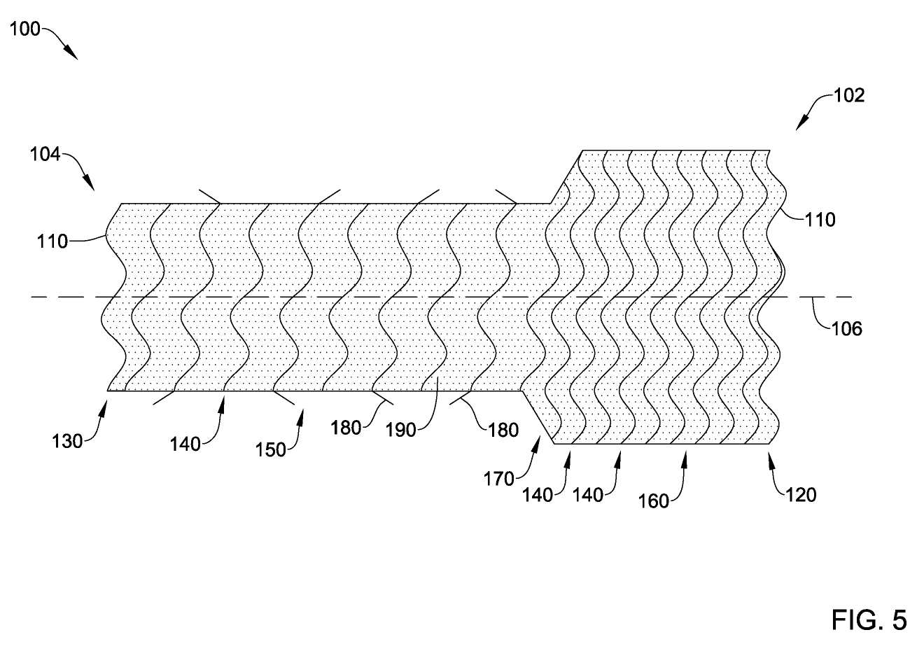

In some embodiments, the plurality of circumferential segments 140 may have a first density within the body portion 150 and a second density within the flared portion 160 and/or the transition portion 170. In at least some embodiments, the second density may be greater than the first density, as seen in FIG. 5. In some embodiments, the second density being greater than the first density may lead to, cause, and/or result in the flared portion 160 and/or the transition portion 170 having a higher radial force than the body portion 150. In some embodiments, the higher radial force may increase resistance to migration downstream and/or toward the second end 104. In some embodiments, the plurality of circumferential segments 140 may have a density that varies gradually along the overall length of the endoprosthesis 100. In some embodiments, the plurality of circumferential segments 140 may have a density that varies within the body portion 150 and/or within the flared portion 160 and/or the transition portion 170. In some embodiments, the plurality of circumferential segments 140 may have a density that varies at different rates within the body portion 150 and the flared portion 160 and/or the transition portion 170. Other configurations are also contemplated.

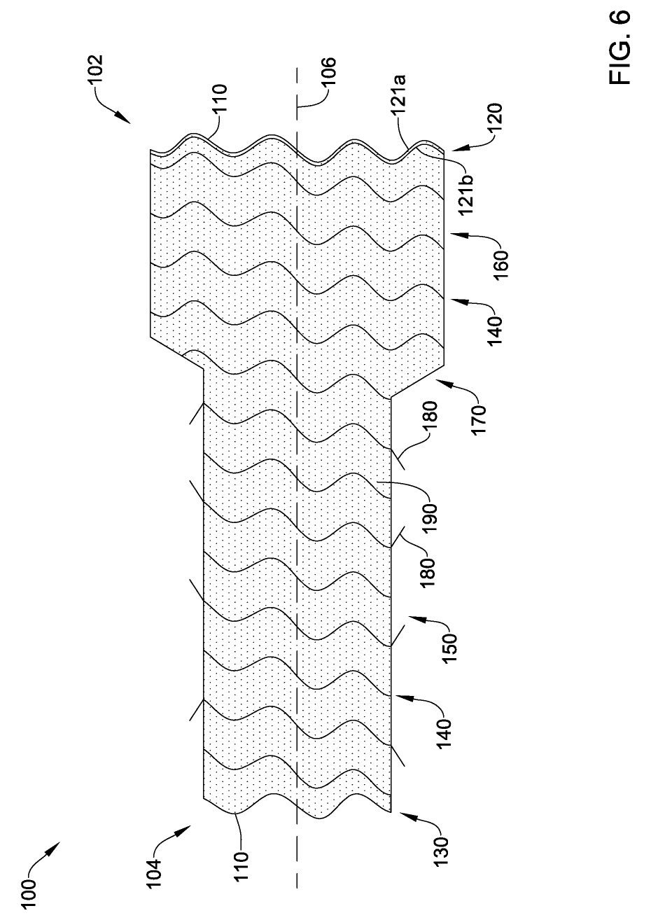

In some embodiments, the first circumferential end segment 120 may comprise a double circumferential loop of the single wire 110, as seen in FIG. 6. In some embodiments, the first circumferential end segment 120, and/or the double circumferential loop thereof, may comprise a first circumferential loop 121a formed from the single wire 110 and a second circumferential loop 121b formed from the single wire 110, wherein the first circumferential loop 121a of the first circumferential end segment 120 and the second circumferential loop 121b of the first circumferential end segment 120 extend circumferentially around the central longitudinal axis 106 in immediate proximity to each other. In some embodiments, the first circumferential loop 121a of the first circumferential end segment 120 may be oriented substantially perpendicular to the central longitudinal axis 106. In some embodiments, the second circumferential loop 121b of the first circumferential end segment 120 may be oriented substantially perpendicular to the central longitudinal axis 106. In some embodiments, the first circumferential loop 121a of the first circumferential end segment 120 may be oriented substantially parallel to the second circumferential loop 121b of the first circumferential end segment 120. In some embodiments, the first circumferential loop 121a and the second circumferential loop 121b of the first circumferential end segment 120 may extend circumferentially around the central longitudinal axis 106 in direct contact with other. In some embodiments, the first circumferential loop 121a of the first circumferential end segment 120 may be fixedly secured directly to the second circumferential loop 121b of the first circumferential end segment 120, such as by welding, soldering, brazing, or adhesive. In some embodiments, the double circumferential loop of the first circumferential end segment 120 may add strength and/or support to the first end 102 of the endoprosthesis 100 to facilitate removal of the endoprosthesis 100 from the patient after deployment. Other benefits and/or advantages (e.g., preventing radial collapse, improved migration resistance, providing a larger target for removal (e.g., via grasper, forceps, or other methods) etc.) are also contemplated.

While not expressly shown, in some embodiments, the second circumferential end segment 130 may comprise a double circumferential loop of the single wire 110. In some embodiments, the second circumferential end segment 130, and/or the double circumferential loop thereof, may comprise a first circumferential loop formed from the single wire 110 and a second circumferential loop formed from the single wire 110, wherein the first circumferential loop and the second circumferential loop of the second circumferential end segment 130 extend circumferentially around the central longitudinal axis 106 in immediate proximity to each other. In some embodiments, the first circumferential loop of the second circumferential end segment 130 may be oriented substantially perpendicular to the central longitudinal axis 106. In some embodiments, the second circumferential loop of the second circumferential end segment 130 may be oriented substantially perpendicular to the central longitudinal axis 106. In some embodiments, the first circumferential loop of the second circumferential end segment 130 may be oriented substantially parallel to the second circumferential loop of the second circumferential end segment 130. In some embodiments, the first circumferential loop and the second circumferential loop of the second circumferential end segment 130 may extend circumferentially around the central longitudinal axis 106 in direct contact with other. In some embodiments, the first circumferential loop of the second circumferential end segment 130 may be fixedly secured directly to the second circumferential loop of the second circumferential end segment 130, such as by welding, soldering, brazing, or adhesive. In some embodiments, the double circumferential loop of the second circumferential end segment 130 may add strength and/or support to the second end 104 of the endoprosthesis 100 to prevent radial collapse of the second end 104 after deployment. In some embodiments, the polymeric covering 190 may be configured such that the second circumferential end segment 130 is uncovered with the plurality of circumferential segments 140 being covered to allow for potential ingrowth of tissue into and/or around the second circumferential end segment 130 to further improve migration resistance or prevention.

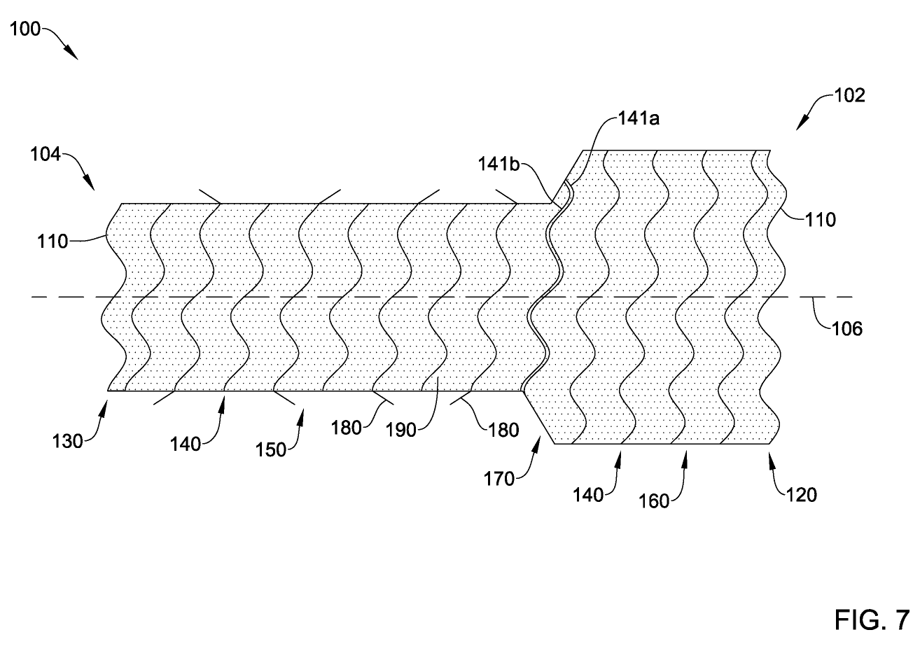

In some embodiments, at least one circumferential segment of the plurality of circumferential segments 140 may comprise a double circumferential loop of the single wire 110, as seen in FIG. 7. In some embodiments, at least one circumferential segment of the plurality of circumferential segments 140 disposed within the transition portion 170 may comprise a double circumferential loop of the single wire 110. In some embodiments, the at least one circumferential segment of the plurality of circumferential segments 140, and/or the double circumferential loop thereof, may comprise a first circumferential loop 141a formed from the single wire 110 and a second circumferential loop 141b formed from the single wire 110, wherein the first circumferential loop 141a and the second circumferential loop 141b of the at least one circumferential segment of the plurality of circumferential segments 140 extend circumferentially around the central longitudinal axis 106 in immediate proximity to each other. In some embodiments, the first circumferential loop 141a of the at least one circumferential segment of the plurality of circumferential segments 140 may be oriented substantially perpendicular to the central longitudinal axis 106. In some embodiments, the second circumferential loop 141b of the at least one circumferential segment of the plurality of circumferential segments 140 may be oriented substantially perpendicular to the central longitudinal axis 106. In some embodiments, the first circumferential loop 141a of the at least one circumferential segment of the plurality of circumferential segments 140 may be oriented substantially parallel to the second circumferential loop 141b of the at least one circumferential segment of the plurality of circumferential segments 140. In some embodiments, the first circumferential loop141a and the second circumferential loop 141b of the at least one circumferential segment of the plurality of circumferential segments 140 may extend circumferentially around the central longitudinal axis 106 in direct contact with other. In some embodiments, the first circumferential loop 141a of the at least one circumferential segment of the plurality of circumferential segments 140 may be fixedly secured directly to the second circumferential loop 141b of the at least one circumferential segment of the plurality of circumferential segments 140, such as by welding, soldering, brazing, or adhesive. In some embodiments, the double circumferential loop of the at least one circumferential segment of the plurality of circumferential segments 140 may add strength and/or support to the body portion 150, the flared portion 160, and/or the transition portion 170 of the endoprosthesis 100 (e.g., to the portion(s) where the double circumferential loop is located) to facilitate removal of the endoprosthesis 100 from the patient after deployment. Other benefits and/or advantages (e.g., preventing radial collapse, migration resistance, providing more robust reinforcement when the endoprosthesis 100 is being removed (e.g., via grasper, forceps, or other methods), providing division between the flared portion 160 and the body portion 150, etc.) are also contemplated.

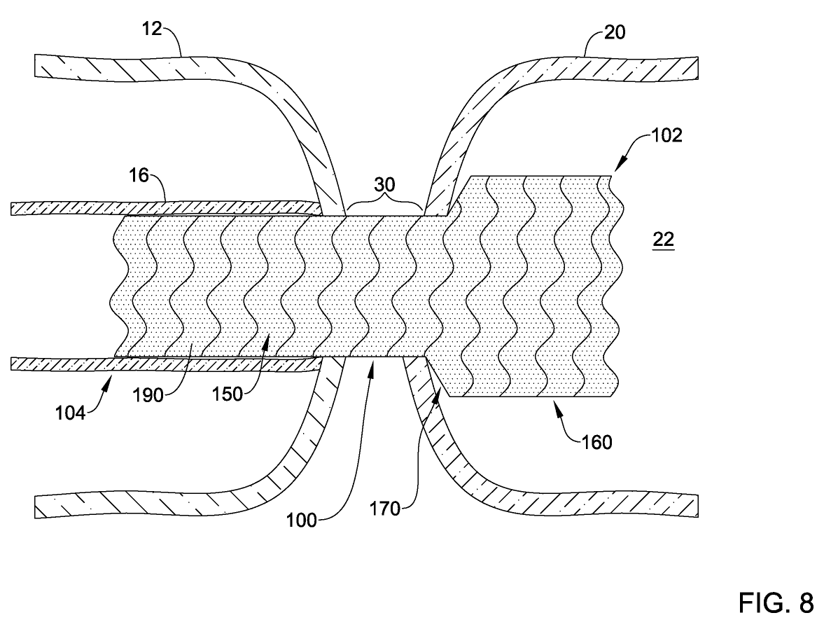

In some embodiments, the body portion 150 of the endoprosthesis 100 may be adapted, sized, and/or configured to be disposed within a first body lumen and the flared portion 160 of the endoprosthesis 100 may be adapted, sized, and/or configured to be disposed within a second body lumen, as seen in FIG. 8. In at least some embodiments, the first body lumen may be a duct of the biliary system 10 (e.g., the hepatic ducts 16) of a patient. In at least some embodiments, the second body lumen may be the stomach 20 of the patient. In some embodiments, the body portion 150 of the endoprosthesis 100 may be adapted, sized, and/or configured to extend through a wall of the second body lumen (e.g., the stomach 20) and/or through a wall of the first body lumen (e.g., the duct of the biliary system 10, the hepatic ducts 16, etc.).

In some embodiments, the flared portion 160 of the endoprosthesis 100 may be adapted, sized, and/or configured to extend and/or to project into an interior space 22 formed within the second body lumen (e.g., the stomach 20) such that the first end 102 of the endoprosthesis 100 is spaced apart from the wall of the second body lumen (e.g., the stomach 20). In some embodiments, the transition portion 170 of the endoprosthesis 100 may be adapted, sized, and/or configured to be engaged with and/or positioned against the wall of the second body lumen (e.g., the stomach 20). In some embodiments, the transition portion 170 and/or the flared portion 160 may be adapted, sized, and/or configured to prevent migration of the endoprosthesis 100 toward the first body lumen (e.g., the duct of the biliary system 10, the hepatic ducts 16, etc.) and/or out of the second body lumen (e.g., the stomach 20).

In some embodiments, the body portion 150 of the endoprosthesis 100 may be adapted, sized, and/or configured to span a gap 30 (e.g., the peritoneal space) between the first body lumen (e.g., the duct of the biliary system 10) and the second body lumen (e.g., the stomach 20). As discussed herein, the endoprosthesis 100 may be fully covered by the polymeric covering 190. The polymeric covering 190 may enable the endoprosthesis 100 to provide a fluid-tight lumen and/or conduit between the first body lumen (e.g., the duct of the biliary system 10) and the second body lumen (e.g., the stomach 20). Additionally, the polymeric covering 190 may prevent leakage within and/or into the gap 30 (e.g., the peritoneal space) between the first body lumen (e.g., the duct of the biliary system 10) and the second body lumen (e.g., the stomach 20), thereby protecting adjacent tissues and/or anatomy.

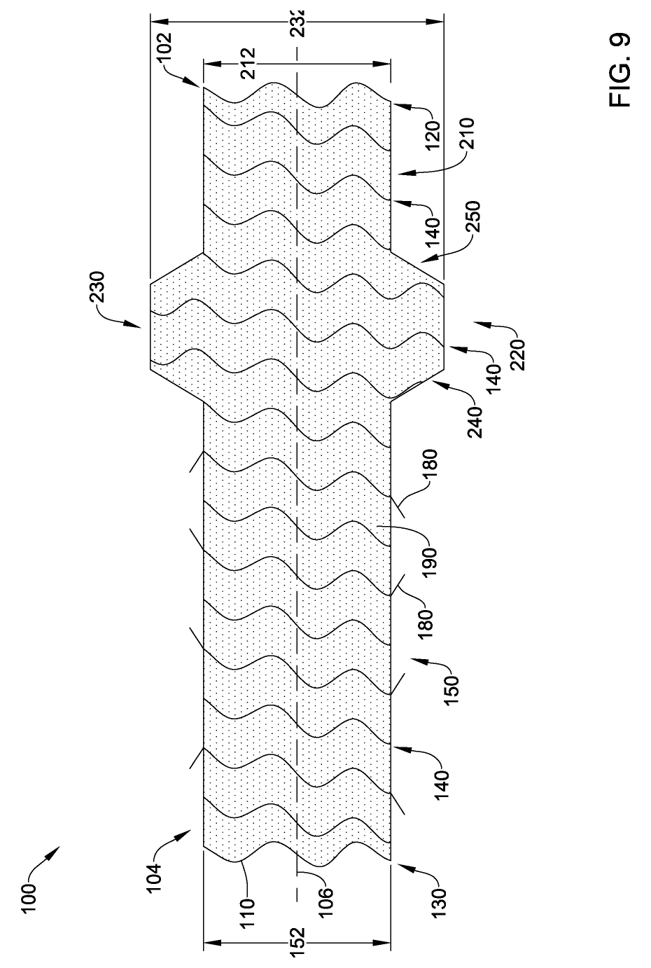

FIG. 9 schematically illustrates selected aspects of an example configuration of the endoprosthesis 100 in a side view. It will be appreciated that many, if not all, of the features, characteristics, benefits, advantages, etc. of the endoprosthesis 100 illustrated in FIGS. 2-7 may also apply to the endoprosthesis 100 of FIG. 9. Accordingly, like reference numerals are used to refer to like elements.

Similar to the endoprosthesis 100 described above, the endoprosthesis 100 of FIG. 9 may comprise a body portion 150 extending axially along the central longitudinal axis 106 from the second end 104 toward the first end 102. The endoprosthesis 100 may comprise a first end portion 210 extending axially along the central longitudinal axis 106 from the first end 102 toward the second end 104. The endoprosthesis 100 may comprise a flanged portion 220 extending between the body portion 150 and the first end portion 210. In some embodiments, the body portion 150, the first end portion 210, and the flanged portion 220 may be formed from the single wire 110 extending from the first end 102 to the second end 104. As discussed herein, the single wire 110 may form the first circumferential end segment 120, the second circumferential end segment 130, and the plurality of circumferential segments 140 extending helically around the central longitudinal axis 106 between the first circumferential end segment 120 and the second circumferential end segment 130. Accordingly, the flanged portion 220 may be formed from the plurality of circumferential segments 140.

The flanged portion 220 may comprise a medial region 230 having a medial outer diameter 232, wherein the medial outer diameter 232 is greater than an outer diameter of the body portion 150 (e.g., the first outer diameter 152) and the medial outer diameter 232 is greater than an outer diameter 212 of the first end portion 210. In some embodiments, the outer diameter 212 of the first end portion 210 may be similar to the outer diameter of the body portion 150 (e.g., the first outer diameter 152). In some embodiments, the outer diameter 212 of the first end portion 210 may be equal to the outer diameter of the body portion 150 (e.g., the first outer diameter 152). In some embodiments, the outer diameter 212 of the first end portion 210 may be within about 10% of the outer diameter of the body portion 150 (e.g., the first outer diameter 152). In some embodiments, the outer diameter 212 of the first end portion 210 may be within about 5% of the outer diameter of the body portion 150 (e.g., the first outer diameter 152). Other configurations are also contemplated.

In some embodiments, the medial outer diameter 232 may be about 20% greater than the outer diameter of the body portion 150 (e.g., the first outer diameter 152) and/or the outer diameter 212 of the first end portion 210. In some embodiments, the medial outer diameter 232 may be about 30% greater than the outer diameter of the body portion 150 (e.g., the first outer diameter 152) and/or the outer diameter 212 of the first end portion 210. In some embodiments, the medial outer diameter 232 may be about 40% greater than the outer diameter of the body portion 150 (e.g., the first outer diameter 152) and/or the outer diameter 212 of the first end portion 210. In some embodiments, the medial outer diameter 232 may be about 50% greater than the outer diameter of the body portion 150 (e.g., the first outer diameter 152) and/or the outer diameter 212 of the first end portion 210. Other configurations are also contemplated. In some embodiments, the medial outer diameter 232 may be between about 2.2 millimeters and about 45 millimeters, between about 4.8 millimeters and about 37.5 millimeters, between about 7.2 millimeters and about 30 millimeters, between about 9.6 millimeters and about 22.5 millimeters, etc. In one non-limiting example, the medial outer diameter 232 may be about 14 millimeters. Other configurations are also contemplated.

In some embodiments, the outer diameter 212 of the first end portion 210 being smaller than the medial outer diameter 232 may reduce food impaction on the first end 102 of the endoprosthesis 100 while permitting and/or maintaining internal biliary drainage.

The flanged portion 220 may comprise a first transition portion 240 extending from the body portion 150 to the medial region 230. The first transition portion 240 may angle radially outward from the body portion 150 toward and/or to the medial region 230 in the deployed configuration of the endoprosthesis 100. The flanged portion 220 may comprise a second transition portion 250 extending from the first end portion 210 to the medial region 230. The second transition portion 250 may angle radially outward from the first end portion 210 toward and/or to the medial region 230 in the deployed configuration of the endoprosthesis 100.

The first transition portion 240 and the second transition portion 250 may be formed from the single wire 110. In some embodiments, the first transition portion 240 may comprise at least one circumferential segment of the plurality of circumferential segments 140. In some embodiments, the first transition portion 240 may comprise two or more circumferential segments of the plurality of circumferential segments 140. In some embodiments, the second transition portion 250 may comprise at least one circumferential segment of the plurality of circumferential segments 140. In some embodiments, the second transition portion 250 may comprise two or more circumferential segments of the plurality of circumferential segments 140.

As discussed herein, at least one circumferential segment of the plurality of circumferential segments 140 may comprise an anti-migration element 180 projecting radially outward from the at least one circumferential segment of the plurality of circumferential segments 140 and/or an outer surface of the endoprosthesis 100 and/or the body portion 150. The one or more anti-migration elements and/or the anti-migration element 180 of the at least one circumferential segment of the plurality of circumferential segments 140 may be formed from the single wire 110.

Similarly, the endoprosthesis 100 may comprise a polymeric covering 190, shown in the figures via dotted shading, coupled to the body portion 150, the first end portion 210, and the flanged portion 220. In some embodiments, the polymeric covering 190 may be fixedly attached to the body portion 150, the first end portion 210, and the flanged portion 220, and/or the single wire 110, and/or the plurality of circumferential segments 140. In some embodiments, the polymeric covering 190 may extend along an inner surface of the endoprosthesis 100, the single wire 110, and/or the body portion 150, the first end portion 210, and the flanged portion 220. In some embodiments, the polymeric covering 190 may extend along an outer surface of the endoprosthesis 100, the single wire 110, and/or the body portion 150, the first end portion 210, and the flanged portion 220. In at least some embodiments, the single wire 110, and/or the body portion 150, the first end portion 210, and the flanged portion 220 may be embedded within the polymeric covering 190. In some embodiments, the anti-migration element 180, the plurality of anti-migration elements, etc. may be covered by and/or embedded within the polymeric covering 190. Other configurations, including combinations thereof, are also contemplated.

While not expressly illustrated, in some embodiments, the endoprosthesis 100 may comprise a removal suture 200 (e.g., FIG. 4) disposed proximate the first end 102. The removal suture 200 may be configured to facilitate and/or aid removal of the endoprosthesis 100 from the patient after deployment. In some embodiments, the removal suture 200 may be interwoven with the first end 102 and/or the first circumferential end segment 120. In some embodiments, the removal suture 200 may be at least partially wrapped around the single wire 110 within the first circumferential end segment 120. In some embodiments, the removal suture 200 may be configured to be grasped and/or held, such as with a forceps or other grasping tool. In some embodiments, the removal suture 200 may be configured to transfer tension applied thereto to the endoprosthesis 100, the first end 102, and/or the first circumferential end segment 120 to facilitate removing the endoprosthesis 100 from the patient after deployment. In some embodiments, the removal suture 200 may be partially embedded within the polymeric covering 190. In some embodiments, the removal suture 200 may be passed through the polymeric covering 190 as it is interwoven with the first circumferential end segment 120. In some embodiments, the removal suture 200 may also be interwoven with one or more circumferential segments of the plurality of circumferential segments 140. Other configurations are also contemplated.

In some embodiments, the plurality of circumferential segments 140 may have a first density within the body portion 150 and/or the first end portion 210, and a second density within the flanged portion 220, the first transition portion 240, and/or the second transition portion 250. In at least some embodiments, the second density may be greater than the first density. In some embodiments, the second density being greater than the first density may lead to, cause, and/or result in the flanged portion 220, the first transition portion 240, and/or the second transition portion 250 having a higher radial force than the body portion 150 and/or the first end portion 210. In some embodiments, the higher radial force may increase resistance to migration downstream and/or toward the second end 104. In some embodiments, the plurality of circumferential segments 140 may have a density that varies gradually along the overall length of the endoprosthesis 100. In some embodiments, the plurality of circumferential segments 140 may have a density that varies within the body portion 150 and/or within the flanged portion 220, the first transition portion 240, and/or the second transition portion 250. In some embodiments, the plurality of circumferential segments 140 may have a density that varies at different rates within the body portion 150 and the flanged portion 220, the first transition portion 240, and/or the second transition portion 250. Other configurations are also contemplated.

In some embodiments, the first circumferential end segment 120 may comprise a double circumferential loop of the single wire 110, as described herein. In some embodiments, the second circumferential end segment 130 may comprise a double circumferential loop of the single wire 110, as described herein. In some embodiments, at least one circumferential segment of the plurality of circumferential segments 140 may comprise a double circumferential loop of the single wire 110, as described herein. In some embodiments, at least one circumferential segment of the plurality of circumferential segments 140 disposed within the flanged portion 220, the first transition portion 240, and/or the second transition portion 250 may comprise a double circumferential loop of the single wire 110.

In some embodiments, the body portion 150 of the endoprosthesis 100 may be adapted, sized, and/or configured to be disposed within a first body lumen, and the first end portion 210 and the flanged portion 220 of the endoprosthesis 100 may be adapted, sized, and/or configured to be disposed within a second body lumen. In at least some embodiments, the first body lumen may be a duct of the biliary system 10 (e.g., the hepatic ducts 16) of a patient. In at least some embodiments, the second body lumen may be the stomach 20 of the patient. In some embodiments, the body portion 150 of the endoprosthesis 100 may be adapted, sized, and/or configured to extend through a wall of the second body lumen (e.g., the stomach 20) and/or through a wall of the first body lumen (e.g., the duct of the biliary system 10, the hepatic ducts 16, etc.).

In some embodiments, the first end portion 210 of the endoprosthesis 100 may be adapted, sized, and/or configured to extend and/or to project into an interior space 22 formed within the second body lumen (e.g., the stomach 20) such that the first end 102 of the endoprosthesis 100 is spaced apart from the wall of the second body lumen (e.g., the stomach 20). In some embodiments, the flanged portion 220 and/or the first transition portion 240 of the endoprosthesis 100 may be adapted, sized, and/or configured to be engaged with and/or positioned against the wall of the second body lumen (e.g., the stomach 20). In some embodiments, the flanged portion 220 and/or the first transition portion 240 may be adapted, sized, and/or configured to prevent migration of the endoprosthesis 100 toward the first body lumen (e.g., the duct of the biliary system 10, the hepatic ducts 16, etc.) and/or out of the second body lumen (e.g., the stomach 20).

In some embodiments, the body portion 150 of the endoprosthesis 100 may be adapted, sized, and/or configured to span the gap 30 (e.g., FIG. 8) between the first body lumen (e.g., the duct of the biliary system 10) and the second body lumen (e.g., the stomach 20). As discussed herein, the endoprosthesis 100 may be fully covered by the polymeric covering 190. The polymeric covering 190 may enable the endoprosthesis 100 to provide a fluid-tight lumen and/or conduit between the first body lumen (e.g., the duct of the biliary system 10) and the second body lumen (e.g., the stomach 20). Additionally, the polymeric covering 190 may prevent leakage within and/or into the gap 30 (e.g., the peritoneal space) between the first body lumen (e.g., the duct of the biliary system 10) and the second body lumen (e.g., the stomach 20), thereby protecting adjacent tissues and/or anatomy.

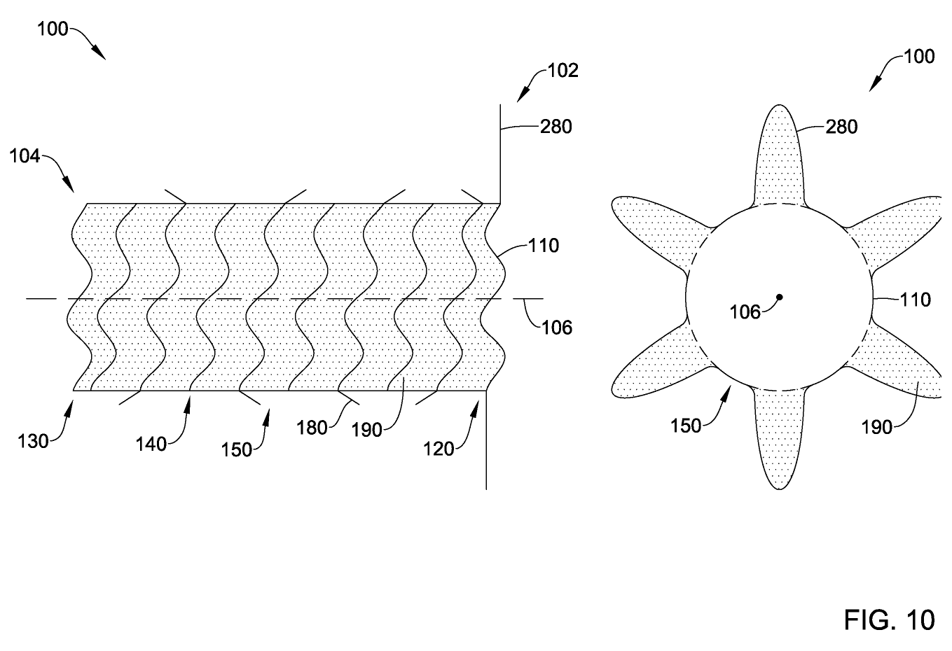

FIG. 10 schematically illustrates selected aspects of an example configuration of the endoprosthesis 100 in a side view. It will be appreciated that many, if not all, of the features, characteristics, benefits, advantages, etc. of the endoprosthesis 100 illustrated in FIGS. 2-7 may also apply to the endoprosthesis 100 of FIG. 10. Accordingly, like reference numerals are used to refer to like elements.

Similar to the endoprosthesis 100 described above, the endoprosthesis 100 of FIG. 9 may comprise a body portion 150 extending axially along the central longitudinal axis 106 from the first end 102 to the second end 104. The endoprosthesis 100 may comprise a plurality of loops 280 extending radially outward at and/or from the first end 102. In some embodiments, the body portion 150 and the plurality of loops 280 may be formed from the single wire 110 extending from the first end 102 to the second end 104. As discussed herein, the single wire 110 may form the first circumferential end segment 120, the second circumferential end segment 130, and the plurality of circumferential segments 140 extending helically around the central longitudinal axis 106 between the first circumferential end segment 120 and the second circumferential end segment 130.

The plurality of loops 280 may be configured to shift between an aligned delivery configuration (not shown) and a projecting deployed configuration (e.g., FIG. 10). In the aligned delivery configuration, which may correspond to and/or coincide with the delivery configuration of the endoprosthesis 100, the plurality of loops 280 may be oriented generally parallel to the central longitudinal axis 106. For example, in the aligned delivery configuration, the plurality of loops 280 may extend axially from the first end 102 and/or the first circumferential end segment 120. In at least one embodiment, the plurality of loops 280 may extend axially away from the first end 102 and/or the first circumferential end segment 120 in a direction opposite from (e.g., not toward) the second end 104.

In some embodiments, in the projecting deployed configuration, which may correspond to and/or coincide with the deployed configuration of the endoprosthesis 100, the plurality of loops 280 may be oriented generally perpendicular to the central longitudinal axis 106, as seen in FIG. 10. For example, in the projecting deployed configuration, the plurality of loops 280 may extend radially outward from the first end 102 and/or the first circumferential end segment 120 at an angle generally normal and/or perpendicular to the central longitudinal axis 106.

In some alternative embodiments, the plurality of loops 280 may extend radially outward from the first end 102 and/or the first circumferential end segment 120 and away from the second end 104 at an angle relative to the central longitudinal axis 106. In some embodiments, the angle may be between about 45 degrees and about 90 degrees. Other angles and/or configurations are also contemplated.

In some other alternative embodiments, the plurality of loops 280 may extend radially outward from the first end 102 and/or the first circumferential end segment 120 and toward the second end 104 at an angle relative to the central longitudinal axis 106. In some embodiments, the angle may be between about 45 degrees and about 90 degrees. Other angles and/or configurations are also contemplated.

In some embodiments, the plurality of loops 280 may resemble and/or take the form of petals (e.g., like a flower) extending radially outward from the first end 102 and/or the first circumferential end segment 120 in the projecting deployed configuration. In at least some embodiments, the plurality of loops 280 may be circumferentially spaced apart from each other. In some alternative embodiments, it is contemplated that the plurality of loops 280 may circumferentially overlap each other. Other configurations, including combinations thereof, are also contemplated.

As discussed herein, at least one circumferential segment of the plurality of circumferential segments 140 may comprise an anti-migration element 180 projecting radially outward from the at least one circumferential segment of the plurality of circumferential segments 140 and/or an outer surface of the endoprosthesis 100 and/or the body portion 150. The one or more anti-migration elements and/or the anti-migration element 180 of the at least one circumferential segment of the plurality of circumferential segments 140 may be formed from the single wire 110.

Similarly, the endoprosthesis 100 may comprise a polymeric covering 190, shown in the figures via dotted shading, coupled to the body portion 150 and, optionally, the plurality of loops 280. In some embodiments, the polymeric covering 190 may be fixedly attached to the body portion 150 and/or the plurality of loops 280, and/or the single wire 110, and/or the plurality of circumferential segments 140. In some embodiments, the polymeric covering 190 may extend along an inner surface of the endoprosthesis 100, the single wire 110, the body portion 150, and/or the plurality of loops 280. In some embodiments, the polymeric covering 190 may extend along an outer surface of the endoprosthesis 100, the single wire 110, the body portion 150, and/or the plurality of loops 280. In at least some embodiments, the single wire 110, the body portion 150, and/or and the plurality of loops 280 may be embedded within the polymeric covering 190. In some embodiments, the anti-migration element 180, the plurality of anti-migration elements, etc. may be covered by and/or embedded within the polymeric covering 190. Other configurations, including combinations thereof, are also contemplated. In some embodiments, the plurality of loops 280 may be devoid of the polymeric covering 190.

While not expressly illustrated, in some embodiments, the endoprosthesis 100 may comprise a removal suture 200 (e.g., FIG. 4) disposed proximate the first end 102. The removal suture 200 may be configured to facilitate and/or aid removal of the endoprosthesis 100 from the patient after deployment. In some embodiments, the removal suture 200 may be interwoven with the first end 102, the first circumferential end segment 120, and/or the plurality of loops 280. In some embodiments, the removal suture 200 may be at least partially wrapped around the single wire 110 within the first circumferential end segment 120 and/or the plurality of loops 280. In some embodiments, the removal suture 200 may be configured to be grasped and/or held, such as with a forceps or other grasping tool. In some embodiments, the removal suture 200 may be configured to transfer tension applied thereto to the endoprosthesis 100, the first end 102, the first circumferential end segment 120, and/or the plurality of loops 280 to facilitate removing the endoprosthesis 100 from the patient after deployment. In some embodiments, the removal suture 200 may be partially embedded within the polymeric covering 190. In some embodiments, the removal suture 200 may be passed through the polymeric covering 190 as it is interwoven with the first circumferential end segment 120. In some embodiments, the removal suture 200 may also be interwoven with one or more circumferential segments of the plurality of circumferential segments 140. Other configurations are also contemplated.

In some embodiments, the first circumferential end segment 120 and/or the plurality of loops 280 may comprise a double circumferential loop of the single wire 110, as described herein. In some embodiments, the second circumferential end segment 130 may comprise a double circumferential loop of the single wire 110, as described herein. In some embodiments, at least one circumferential segment of the plurality of circumferential segments 140 may comprise a double circumferential loop of the single wire 110, as described herein.

In some embodiments, the body portion 150 of the endoprosthesis 100 may be adapted, sized, and/or configured to be disposed within a first body lumen, and the first end 102 and/or the plurality of loops 280 of the endoprosthesis 100 may be adapted, sized, and/or configured to be disposed within a second body lumen. In at least some embodiments, the first body lumen may be a duct of the biliary system 10 (e.g., the hepatic ducts 16) of a patient. In at least some embodiments, the second body lumen may be the stomach 20 of the patient. In some embodiments, the body portion 150 of the endoprosthesis 100 may be adapted, sized, and/or configured to extend through a wall of the second body lumen (e.g., the stomach 20) and/or through a wall of the first body lumen (e.g., the duct of the biliary system 10, the hepatic ducts 16, etc.).

In some embodiments, the first end 102 of the endoprosthesis 100 may be adapted, sized, and/or configured to open into an interior space 22 formed within the second body lumen (e.g., the stomach 20). In some embodiments, the plurality of loops 280 may be adapted, sized, and/or configured to be engaged with and/or positioned against the wall of the second body lumen (e.g., the stomach 20). In some alternative embodiments, the plurality of loops 280 may be adapted and/or configured to extend and/or angle away from the first end 102 into the interior space 22 formed within the second body lumen (e.g., away from the wall of the stomach 20). In some other alternative embodiments, the plurality of loops 280 may be adapted and/or configured to extend and/or angle away from the first end 102 toward the wall of the stomach 20. In some embodiments, the plurality of loops 280 may be adapted, sized, and/or configured to prevent migration of the endoprosthesis 100 toward the first body lumen (e.g., the duct of the biliary system 10, the hepatic ducts 16, etc.) and/or out of the second body lumen (e.g., the stomach 20).

In some embodiments, the body portion 150 of the endoprosthesis 100 may be adapted, sized, and/or configured to span the gap 30 (e.g., FIG. 8) between the first body lumen (e.g., the duct of the biliary system 10) and the second body lumen (e.g., the stomach 20). As discussed herein, the endoprosthesis 100 may be fully covered by the polymeric covering 190. The polymeric covering 190 may enable the endoprosthesis 100 to provide a fluid-tight lumen and/or conduit between the first body lumen (e.g., the duct of the biliary system 10) and the second body lumen (e.g., the stomach 20). Additionally, the polymeric covering 190 may prevent leakage within and/or into the gap 30 (e.g., the peritoneal space) between the first body lumen (e.g., the duct of the biliary system 10) and the second body lumen (e.g., the stomach 20), thereby protecting adjacent tissues and/or anatomy.

The materials that can be used for the various components of the endoprosthesis and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the endoprosthesis, the single wire, the polymeric covering, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoprosthesis having a first end and a second end, comprising:
a body portion extending axially along a central longitudinal axis; and
a flared portion extending between the body portion and the first end;
wherein the body portion and the flared portion are formed from a single wire extending from the first end to the second end;
wherein the single wire forms:
a first circumferential end segment extending circumferentially around the central longitudinal axis at the first end, wherein the first circumferential end segment is oriented generally perpendicular to the central longitudinal axis;
a second circumferential end segment extending circumferentially around the central longitudinal axis at the second end, wherein the second circumferential end segment is oriented generally perpendicular to the central longitudinal axis; and
a plurality of circumferential segments extending helically around the central longitudinal axis between the first circumferential end segment and the second circumferential end segment.

2. The endoprosthesis of claim 1, further comprising a transition portion extending from the body portion to the flared portion.

3. The endoprosthesis of claim 2, wherein the transition portion angles radially outward from the body portion to the flared portion.

4. The endoprosthesis of claim 2, wherein at least one circumferential segment of the plurality of circumferential segments disposed within the transition portion comprises a double circumferential loop of the single wire.

5. The endoprosthesis of claim 1, wherein the body portion has a first outer diameter, and the flared portion has a second outer diameter greater than the first outer diameter.

6. The endoprosthesis of claim 1, wherein the plurality of circumferential segments is oriented at an oblique angle relative to the central longitudinal axis, as viewed from a side of the endoprosthesis toward the central longitudinal axis.

7. The endoprosthesis of claim 6, wherein the oblique angle is between about 70 degrees and about 85 degrees.

8. The endoprosthesis of claim 1, wherein a removal suture is interwoven with the first circumferential end segment.

9. The endoprosthesis of claim 1, wherein the plurality of circumferential segments has a first density within the body portion and a second density greater than the first density within the flared portion.

10. The endoprosthesis of claim 1, further comprising a polymeric covering fixedly attached to the body portion and the flared portion.

11. The endoprosthesis of claim 1, wherein at least one circumferential segment of the plurality of circumferential segments disposed within the body portion comprises an anti-migration element projecting radially outward from the at least one circumferential segment of the plurality of circumferential segments disposed within the body portion, wherein the anti-migration element is formed from the single wire.

12. The endoprosthesis of claim 1, wherein the first circumferential end segment comprises a double circumferential loop of the single wire.

13. The endoprosthesis of claim 1, wherein the body portion is configured to be disposed within a first body lumen and the flared portion is configured to project into an interior space formed within a second body lumen such that the first end is spaced apart from a wall of the second body lumen.

14. The endoprosthesis of claim 1, wherein the first body lumen is a duct of a biliary system of a patient and the second body lumen is a stomach of the patient.

15. An endoprosthesis having a first end and a second end, comprising:
a body portion extending axially along a central longitudinal axis from the second end toward the first end;
a first end portion extending axially along a central longitudinal axis from the first end toward the second end; and
a flanged portion extending between the body portion and the first end portion;
wherein the body portion, the first end portion, and the flanged portion are formed from a single wire extending from the first end to the second end;
wherein the single wire forms:
a first circumferential end segment extending circumferentially around the central longitudinal axis at the first end, wherein the first circumferential end segment is oriented generally perpendicular to the central longitudinal axis;
a second circumferential end segment extending circumferentially around the central longitudinal axis at the second end, wherein the circumferential second end segment is oriented generally perpendicular to the central longitudinal axis; and
a plurality of circumferential segments extending helically around the central longitudinal axis between the first circumferential end segment and the second circumferential end segment.

16. The endoprosthesis of claim 15, wherein the flanged portion comprises:
a medial region having a medial outer diameter, wherein the medial outer diameter is greater than an outer diameter of the body portion and the medial outer diameter is greater than an outer diameter of the first end portion;
a first transition portion extending from the body portion to the medial region; and
a second transition portion extending from the first end portion to the medial region.

17. The endoprosthesis of claim 15, further comprising a polymeric covering fixedly attached to the body portion, the first end portion, and the flanged portion.

18. The endoprosthesis of claim 17, wherein the body portion is configured to be disposed within a first body lumen and the first end portion is configured to project into an interior space formed within a second body lumen such that the first end is spaced apart from a wall of the second body lumen;
wherein the body portion is configured to span a gap between the first body lumen and the second body lumen.

19. The endoprosthesis of claim 15, wherein at least one circumferential segment of the plurality of circumferential segments disposed within the body portion comprises an anti-migration element projecting radially outward from the at least one circumferential segment of the plurality of circumferential segments disposed within the body portion, wherein the anti-migration element is formed from the single wire.

20. An endoprosthesis having a first end and a second end, comprising:
a body portion extending axially along a central longitudinal axis from the first end to the second end; and
a plurality of loops extending radially outward from the first end;
wherein the body portion and the plurality of loops are formed from a single wire extending from the first end to the second end;
wherein the single wire forms:
a first circumferential end segment extending circumferentially around the central longitudinal axis at the first end, wherein the first circumferential end segment includes the plurality of loops and is oriented generally perpendicular to the central longitudinal axis;
a second circumferential end segment extending circumferentially around the central longitudinal axis at the second end, wherein the second circumferential end segment is oriented generally perpendicular to the central longitudinal axis; and
a plurality of circumferential segments extending helically around the central longitudinal axis between the first circumferential end segment and the second circumferential end segment.
